(19) United States
(12) Patent Application Publication
(10) Pub. No.: US 2002/0098182 A1
(43) Pub. Date: Jul. 25, 2002

(54) Treatment of immune-mediated diseases by oral administration of plasma fractions enriched in immunoglobulin G

(76) Inventors: Richard Weisbart, Los Angeles, CA (US); Leon E. Barstow, Tucson, AZ (US)

Correspondence Address: SCULLY, SCOTT, MURPHY & PRESSER, 400 Garden City Plaza, Garden City, NY, 11530, US

(21) Appl. No.: 09/966,119

(22) Filed: Sep. 28, 2001

Related U.S. Application Data
(60) Provisional application No. 60/236,255, filed on Sep. 28, 2000.

(51) Int. Cl. A61K 39/395

(52) U.S. Cl. 424/131.1

(57) ABSTRACT

Human plasma fractions substantially enriched in human immunoglobulin G, such as Cohn Fraction II+III, and optionally an antacid may be administered orally to patients suffering from a myriad of immune-mediated diseases, including rheumatoid arthritis, to treat the disease condition of those patients. Oral administration of Cohn Fraction II+III results in significant clinical improvement in the level of disease activity in patients with rheumatoid arthritis, for example.

### CROSS-REFERENCE TO RELATED APPLICATION

This application claims priority from U.S. Provisional Application No. 60/236,255, filed Sep. 28, 2000.

### FIELD OF THE INVENTION

The present invention relates to the treatment of immune-mediated diseases, including autoimmune diseases. More particularly, the invention relates to the treatment of rheumatoid arthritis (including polyarticular juvenile rheumatoid arthritis), Still's disease, Sjogrens Syndrome, vasculitis, including Systemic Lupus Erythmatosus, peripheral neuropathy, Raynauds Phenomenon, sensory-neural hearing loss (Meniere's Disease), fibromylagia. The invention also relates to the treatment of spondyloarthopathies including, inflammatory bowel disease (ulcerative colitis, Crohn's disease and mucinous colitis), psoriatic arthritis, Reiter's Syndrome and ankylosing spondylitis, temporal arteritis, polymyalgia rheumatica, agammaglobulinemia and immuno-suppressed patients. In accordance with the present invention immune-mediated diseases are treated by oral administration of a pharmaceutical composition comprising Cohn Fraction II, Cohn Fraction III, or Cohn Fraction II and III.

### BACKGROUND OF THE INVENTION

The present invention relates to a method for the treatment of immune-mediated diseases by administering a pharmaceutical composition comprising human plasma fractions substantially enriched in human immunoglobulin G, for example, Cohn Fraction II+III. Cohn Fraction II+III is derived from pooled human plasma and predominantly contains IgG, IgA and IgM. Cohn Fraction II+III is commonly prepared according to Cohn's method 6, (Cohn E. J., et al. (1946), J. Am. Chem. Soc. 68:459-475 and W.H.O. Technical Report Series 786 (1989), incorporated herein by reference). Cohn Fraction II+III also contains albumin, alpha and beta globulins, glycine, blood clotting factors II, VII, IX and X and dextrose.

The microbial flora of the gastrointestinal tract is believed to have a profound influence on the development of the immune system and predisposition to develop autoimmune diseases. Contamination of the intestine with microbes is essential for the development of systemic immune tolerance to gastrointestinal antigens and the rejection of foreign organ grafts. Gaboriau-Routhiau, et al. (1996), Pediatric Res., 39(4) (1):625-629; Sudo, et al. (1997), J. Immunol. 159(4):1739-1745. The importance of microbes in the development of immune-mediated diseases, including, but not limited to adult and juvenile rheumatoid arthritis, is demonstrated by the relationship between exposure to microbial antigens and the development of HLA-B27 reactive arthritis in humans. Recent studies by Taurog, et al. (1994), J. Exp. Med., 180(6):2359-2364 show that HLA-B27 transgenic rats develop arthritis, while germ free animals do not. Microbes associated with reactive arthritis include those found in the gastrointestinal tract. Similarly, according to several researchers rheumatoid arthritis has a prominent association with HLA-DR4 defined by a shared epitope present on antigens from intestinal microbes such as E. coli, P. mirabilis and Epstein Barr Virus. (Albani, et al. (1992), Clin Biochem., 25(3):209-212; Tiwana, et al. (1999), Infect. Immun., 67(6):2769-2775; Roudier, J., et al., (1989) Proc. Natl. Acad. Sci. USA, 86(13):5104-5108.

Adult rheumatoid arthritis is a systemic inflammatory disease that commonly affects the joints, particularly those of the hands and feet. The onset of rheumatoid arthritis can occur slowly, ranging from a few weeks to a few months, or the condition can surface rapidly in an acute manner. HLA-B27 is associated with the spondyloarthopathies. (Schwimmbeck, et al. (1988) Am. J. Med., 85(6A):51-53; Lahesma, et al., (1991) Clin. Exp. Immunol., 86(3):399-404; Fielder, et al. (1995), FEBS Lett. 369(2-3):243-248; Erbinger, et al. (1996), Clin. Rheum., 1550 Suppl. 1:57-61).

Today, over 2,500,000 individuals have been diagnosed with adult rheumatoid arthritis in the United States alone (1% of population), with some statistics indicating that from 6.5 to 8 million adults are potentially afflicted with the disease. Women are affected 2-3 times more often than men. Adult rheumatoid arthritis can occur in young adults and typically will increase in incidence with age.

The classic early symptoms of adult rheumatoid arthritis include stiffness, tenderness, fever, subcutaneous nodules, achy joints, and fatigue. The joints of the hands, feet, knees and wrists are most commonly affected, with eventual involvement of the hips, elbows and shoulders. As the joints stiffen and swell, any type of motion becomes very painful and difficult. The more severe cases of adult rheumatoid arthritis can lead to intense pain and eventual joint destruction. Some 300,000 bone and joint replacement surgical procedures are performed in the U.S. annually in an effort to alleviate the pain and mobility loss resultant from arthritis related joint destruction.

Adult rheumatoid arthritis and juvenile rheumatoid arthritis are two different diseases. Juvenile rheumatoid arthritis is most common in children and includes eight different forms of disease. One form of juvenile rheumatoid arthritis, Rf-positive polyarticular juvenile rheumatoid arthritis, bears some resemblance to adult rheumatoid arthritis. However, only about 40% of all juvenile rheumatoid arthritis cases are polyarticular and, of these, only about 5-10% are rheumatoid factor (Rf) positive. Therefore, only 2-4% of juvenile rheumatoid arthritis patients suffer from Rf-positive polyarticular juvenile rheumatoid arthritis.

Juvenile rheumatoid arthritis is characterized by abnormal T and B cell function and selective IgA deficiency. Adult rheumatoid arthritis is a disease identified by the presence of auto-antibodies including certain characteristic rheumatoid factors. The immunogenetic associations, clinical course, and functional outcome of juvenile rheumatoid arthritis are quite different from adult-onset rheumatoid arthritis. Pediatric Rheumatic Diseases In: Primer on the Rheumatic Diseases, 11ed. 1997 (incorporated herein by reference).

Adult rheumatoid arthritis is characterized by the presence of rheumatoid factor autoantibodies. Germ free mice genetically predisposed to produce rheumatoid factors do not produce these autoantibodies until such mice are exposed to microbes. After termination of the germ free state, rheumatoid factors are first produced by the lymphoid system of the gastrointestinal tract. Coutelier, et al. (1986) J. Immunol., 137(l):337-340. These observations suggest that in some patients, rheumatoid arthritis is a reactive arthritis induced by microbial antigens in the gastrointestinal tract.

The gastrointestinal tract is protected by the secretory immune system. IgA antibodies are secreted into the intestine in response to microbial antigens. Patients with IgA deficiency have an increased incidence of autoimmune diseases, including reactive arthritis. These observations suggest that immunoglobulin secreted into the intestine protects against autoimmunity. If some individuals fail to produce antibodies that protect against the development of autoimmune diseases, then restoring normal antibodies in the intestine may ameliorate the symptoms of patients with autoimmune disease. To date, the effective treatment of autoimmune diseases such as adult rheumatoid arthritis has generally employed a combination of medication, exercise, rest and proper joint protection therapy. The therapy for a particular patient depends on the severity of the disease and the joints that are involved. Aspirin is widely used for pain and to reduce inflammation. In addition to aspirin, non-steroidal anti-inflammatory drugs, corticosteroids, gold salts, anti-malarials and systemic immuno-suppressants are widely used in moderate to advanced cases. The use of steroids and immunosuppressants, however, has significant risks and side effects both in terms of toxicity and vulnerability to potentially lethal conditions such as infection and malignancy. Thus, there exists a need for a method of treating immune-mediated disease which does not entail the potentially adverse side effects associated with the treatments described above.

“Superantigens” have been considered as stimulants of the immune system in various autoimmune diseases including rheumatoid arthritis. Herman, A., et al. (1991) Annu. Rev. Immunol. 9:745-772; Drake, C. G. and Kotzin, B. L. (1992) J. Clin. Immunol. 12:149-162 (incorporated herein by reference). The gastrointestinal tract may be the site of immunologic stimulation by superantigens. It has been considered that there may be a defect in the ability of patients with adult rheumatoid arthritis to produce antibodies with the correct neutralizing specificities. One approach to treating rheumatoid arthritis is to orally administer cow's milk to patients. See U.S. Pat. No. 4,732,757 (Stolle, et al.). Stolle, et al. disclose that hyperimmunized milk containing a high titer of specific antibodies from animals actively and artificially immunized and boosted with large amounts of purified antigen is useful to treat rheumatoid arthritis. The drawbacks to this approach are several-fold. The cow donor pool must be specifically and actively immunized to a small subset of antigens. In addition, some patients have adverse reactions to consumption of bovine milk. Moreover, cow's milk does not contain the entire spectrum of antibodies present in a human. Furthermore, the effects of hyperimmune milk on inflammatory processes, such as rheumatoid arthritis, has largely been discarded. See Ormrod and Miller (1991) Agents and Actions, 32(3/4):160-166.

Another approach to the treatment of immune-mediated diseases, of which rheumatoid arthritis is an example, is tolerization of the patient suffering from the immune-mediated disease to the particular autoantigen(s) involved in the disease. In Weiner, et al., Science 259:1321-1324 (1993) (incorporated herein by reference), multiple sclerosis patients were orally administered bovine myelin protein, which contains two multiple sclerosis autoantigens. In Trentham, et al., Science 261:1727-1730 (1993), rheumatoid arthritis patients were orally administered collagen, a presumed autoantigen. One drawback to tolerization is that the identification of the correct autoantigen to which tolerance is to be induced is elusive.

In view of the unsuccessful and disadvantageous modalities currently employed to treat those disorders, there is a continued need to develop effective methods and compositions for the treatment of immune-mediated diseases.

### SUMMARY OF THE INVENTION

The present invention is directed to a method for treating an immune-mediated disease by orally administering a composition constituting a human plasma fraction enriched in human immunoglobulin G, such as, Cohn Fraction II, Cohn Fraction III or Cohn Fraction II+III and combinations thereof with or without an antacid in and amount sufficient to provide a clinically observable improvement in a patient's condition. The present invention is based on the surprising discovery that the oral administration of a composition containing immunoglobulin G, optionally in conjunction with an antacid, to patients with immune-mediated disease results in a significant clinical improvement in the condition of the patient. The present invention is also based on the discovery that there are no toxic effects of orally administered immunoglobulin G-enriched compositions that have been irradiated with gamma irradiation, for example.

### DETAILED DESCRIPTION OF THE INVENTION

The present invention provides a method for treating a patient suffering from an immune-mediated disease. By “immune-mediated” disease is meant a pathogenic disease which triggers a characteristic immune response by cells that include lymphocytes, antigen presenting cells and soluble mediators or cytokines produced by said cells. An immune-mediated disease manifests in symptoms such as pain, inflammation, stiffness, hearing loss, and include such diseases as rheumatoid arthritis, Still's disease, Sjorgrens syndrome, and inflammatory bowel disease, for example. The method of the present invention is employed by orally administering a human plasma fraction containing human immunoglobulin G to a subject in need of such plasma fraction.

A preferred human plasma fraction containing human immunoglobulin G is Cohn Fraction II. Another preferred human plasma fraction containing human immunoglobulin G is Cohn Fraction III. Still another preferred human plasma fraction enriched in human immunoglobulin G is Cohn Fraction II+III. The human plasma fraction is administered in accordance with the present invention, optionally in conjunction with an antacid. Cohn Fraction II, Cohn Fraction III, and Cohn Fraction II+III are derived from pooled human plasma and predominantly contain IgG, IgA and IgM. Cohn Fraction II, Cohn Fraction III and Cohn Fraction II+III are each conventionally prepared, and are understood, in accordance with the present invention, to be pooled human immunoglobulin compositions.

An immunoglobulin, introduced into the acidic environment of the human stomach, may suffer inactivation. To alleviate such inactivation and provide increased therapeutic efficacy, the human plasma fraction employed in the methods of the present invention is optionally administered in conjunction with an antacid. While not wishing to be bound to a particular mechanism, the acid blocker may neutralize the otherwise acidic character of the gut thereby shielding the immunoglobulin from digestion in the stomach. Alternatively, the acid-blocker and immunoglobulin may synergistically provide remediation of disease symptoms by suppressing inflammatory mediators or immune-mediated inflammation.

The present invention also contemplates pharmaceutical compositions comprising human plasma fractions such as, for example, Cohn Fraction II, Cohn Fraction III or Cohn Fraction II+III and combinations thereof with or without an antacid.

AS used herein, the term “pooled human immunoglobulin” refers to an immunoglobulin composition containing polyclonal antibodies obtained from the plasma of thousands of human donors. The polyclonal antibodies may include IgG, IgA, IgM, etc. or fragments thereof. A preferred polyclonal fraction contains IgG for treating immune-mediated diseases including rheumatoid arthritis, for example. A preferred immunoglobulin composition, Cohn Fraction II+III, contains at least about 30% to about 85% IgG polyclonal antibodies, about 5% to about 30% IgA and about 1% to about 25% IgM and trace amounts of other components such as, for example, clotting factors II, VII, IX, X and alpha and beta globulins. Another preferred immunoglobulin composition, Cohn Fraction II, contains about 95% to about 99% IgG polyclonal antibodies, at least 0.01% to about 2% IgM and trace amounts of salt. Still another preferred immunoglobulin composition, Cohn Fraction III, contains at least about 25% IgG polyclonal antibodies, at least about 5% to about 30% IgA and about 1% to about 25% IgM, together with trace amounts of clotting factors II, VII, IX, alpha and beta globins and lipids.

A preferred pooled human immunoglobulin composition useful in accordance with the present invention comprises Cohn Fraction II. Another preferred human immunoglobulin composition useful in accordance with the present invention comprises Cohn Fraction III. Still another preferred human immunoglobulin composition useful in accordance with the present invention comprises Fraction II+III.

“Antacid” when used herein denotes an H₂-blocker or acid blocker or other acid neutralizing agent which neutralizes and/or significantly reduces the acidic content of the gut. A preferred antacid useful in accordance with the teachings of the present invention is cimetidine.

A “clinically observable improvement” when used herein refers to a significant subjective remediation of symptoms associated with the patient's immune-mediated condition. For example, in the case of a patient suffering from rheumatoid arthritis, subjective remediation is characterized, in accordance with the present invention as including, but not limited to, tender joint(s), swollen joint(s) and stiffness reduction or amelioration assessments. Significant subjective remediation of symptoms denotes a patient's self-assessment or a physician's assessment of stiffness, joint tenderness, swelling and the like. For example, an observable difference in swelling or tenderness in even one arthritic joint is significant. Absence of swelling or tenderness in a previously affected joint is most significant. Likewise, renewed freedom of movement in a joint(s) previously encumbered by an immune-mediated disease is significant.

Another aspect of the present invention provides a pharmaceutical composition comprising Cohn Fraction II, Cohn Fraction III or Cohn Fraction II+III, optionally an antacid and a pharmaceutically acceptable carrier. In a preferred embodiment the composition comprises Cohn Fraction II+III and a pharmaceutically acceptable carrier.

As used herein, a “pharmaceutically acceptable carrier” includes any and all solvents, dispersion media, coatings, antibacterial and antifungal agents, isotonic and absorption delaying agents, and the like. Some examples of substances which can serve as pharmaceutical carriers are sugars, such as lactose, glucose and sucrose; starches such as corn starch and potato starch; cellulose and its derivatives such as sodium carboxymethylcellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil, corn oil and oil of theobroma; polyols such as propylene glycol, glycerine, sorbitol, manitol, and polyethylene glycol; agar; alginic acids; pyrogen-free water; isotonic saline; and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tableting agents, stabilizers, anti-oxidants and preservatives, can also be present. The use of such media and agents for pharmaceutically active substances is well known in the art. Except insofar as any conventional media or agent is incompatible with the active ingredients, its use in the therapeutic compositions is contemplated.

Accordingly, in a preferred form of treating immune-mediated disease, the patient is orally administered a therapeutically effective amount of Cohn Fraction II, Cohn Fraction III or Cohn Fraction II+III and combinations thereof and a pharmaceutically acceptable carrier. In another preferred form of treating immune-mediated disease the patient is orally administered a therapeutically effective amount of Cohn Fraction II and a pharmaceutically acceptable carrier. In still another preferred form of treating immune-mediated disease the patient is orally administered a therapeutically effective amount of Cohn Fraction III and a pharmaceutically acceptable carrier.

“Treating” or “treatment” as used herein means to ameliorate, suppress, mitigate or eliminate the clinical symptoms after the onset (i.e., clinical manifestation) of an autoimmune disease, such as, for example, rheumatoid arthritis. An effective or successful treatment provides a clinically observable improvement.

“Oral” administration as used herein includes oral, enteral or intragastric administration.

“In conjunction with” as used herein means before, substantially simultaneously with or after oral administration of antacid. Of course, the administration of a composition such as, for example, Cohn Fraction II+III can not precede or follow administration of an antacid by so long an interval of time that the relevant effects of the substance administered first have expired. Therefore, the immunoglobulin composition should usually be administered within a therapeutically effective time. By “therapeutically effective time,” as used herein, is meant a time frame in which the antacid or immunoglobulin composition (e.g., Cohn Fraction II+III or Cohn Fraction II) is still active within the patient.

In a preferred embodiment, the immunoglobulin composition (i.e., Cohn Fraction II, Cohn Fraction III and Cohn Fraction II+III) is produced by cold alcohol (e.g., ethanol) fractionation from the plasma of about 1000 to about 3000 human volunteers according to the Cohn's method 6 (Fraction II+III) supra, and the method of Oncley, et al. (Cohn Fraction II and Fraction III) infra., and incorporated herein by reference.

In order to enhance the effectiveness of the introduced immunoglobulin in the treated patient and provide a clinically observable improvement, an antacid is optionally administered in conjunction with the Cohn Fraction II, Cohn Fraction III, and/or Cohn Fraction II+III composition. In a preferred embodiment the immunoglobulin composition and the antacid are administered simultaneously in a unitary pharmaceutical composition. In another preferred embodiment the immunoglobulin composition is administered at a therapeutically effective time after administration of the antacid. Preferably, the antacid is aluminum hydroxide or magnesium hydroxide such as Maalox®, Mylanta® or Tagamet® which are available commercially. Most preferably the antacid is an H2 blocker, such as Cimetidine or Ranitidine.

The dosage of antacid administered in conjunction with the immunoglobulin composition depends on the particular H₂-blocker used. When the antacid is Mylanta®, between 15 ml and 30 ml is preferred. Most preferably the dosage of Mylanta® is 15 ml. When the cimetidine H2 blocker is used, the preferred dosage is between 400 and 800 mg per day.

The dosage of the immunoglobulin compositions of the present invention administered to the patient may be varied depending upon severity of the patient's condition and other clinical factors. Preferably, the dosage will be as small as possible while still providing a clinically observable and therapeutically effective result. The most preferable and therapeutically effective doses are those that have the largest effect in terms of alleviating the patient's disease condition; including pain. Therapeutically effective dosages of the Cohn Fraction II, Cohn Fraction III and/or Cohn Fraction II+III composition may range from as little as 5 mg/kg up to as much as 5 g/kg per day. For example, for juvenile arthritis patients appropriate doses of the compositions of the present invention are about 5 mg/kg body weight to about 30 mg/kg body weight per day.

Although the preferred dose is given in increments, it may also be given as a single dose. Further, although the dose of the immunoglobulin composition may be administered at any time during the day, it is preferred that it be administered in the morning, prior to substantial patient activity.

In the treatment of rheumatoid arthritis with Cohn Fraction II, Cohn Fraction III, or Cohn Fraction II+III, the patient's arthritic condition can be determined, for example, by the patient's self-assessment of his or her pain, stiffness, etc. Another way to determine the patient's arthritic condition is for a physician to examine a patient's joint tenderness and swelling.

A decided practical advantage of the present invention is that the composition containing a human plasma fraction enriched in human immunoglobulin G, such as, for example, Cohn Fraction II+III, may be administered in a convenient manner such as by the oral route, although the invention also contemplates administering of the claimed compositions by intravenous, aerosol or suppository routes. Oral administration is most preferred. Depending on the route of administration, the active ingredients which comprise the requisite immunoglobulin composition (i.e., Cohn Fraction II, Cohn Fraction III or Cohn Fraction II+III) may be required to be coated in a material to protect said fraction from the action of enzymes, acids and other natural conditions which may adversely affect the active fraction. In order to administer the disclosed compositions orally, such compositions can be coated by, or administered with, a material to prevent inactivation. For example, an enteric coated composition can be specifically designed to transport Cohn Fraction II+III to the gastrointestinal tract. Enteric coating technology is conventional in the art of pharmaceutical preparation and is readily practiced in accordance with the present invention with the knowledge of the ordinarily skilled artisan.

The active compound may be orally administered, for example, with an inert diluent or with an assimilable edible carrier, or it may be enclosed in hard or soft shell gelatin capsule, or it may be compressed into tablets designed to pass through the stomach (i.e., enteric coated), or it may be incorporated directly with the food of the diet. For oral therapeutic administration, Cohn Fraction II, Cohn Fraction III or Cohn Fraction II+III may be incorporated with excipients and used in the form of ingestible tablets, buccal tablets, troches, capsules, elixirs, suspensions, syrups, wafers, and the like.

The tablets, troches, pills, capsules, and the like, as described above, may also contain the following: a binder such as gum tragacanth, acacia, corn starch or gelatin; excipients such as dicalcium phosphate; a disintegrating agent such as corn starch, potato starch, alginic acid, and the like; a lubricant such as magnesium stearate; and a sweetening agent such as sucrose, lactose or saccharin may be added or a flavoring agent such as peppermint, oil or wintergreen or cherry flavoring. When the dosage unit form is a capsule, it may contain, in addition to materials of the above type, a liquid carrier. Various other materials may be present as coatings or to otherwise modify the physical form of the dosage unit. For instance, tablets, pills or capsules may be coated with shellac, sugar or both. A syrup or elixir may contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, Cohn Fraction II, Cohn Fraction III and/or Cohn Fraction II+III may be incorporated into sustained-release preparations and formulations.

It is especially advantageous to formulate the immunoglobulin compositions of the present invention in dosage unit form for ease of administration and uniformity of dosage. “Dosage unit form” as used herein refers to physically discrete units suited as unitary dosages for the subjects to be treated, each unit containing a predetermined quantity of Cohn Fraction II, Cohn Fraction III or Cohn Fraction II+III with or without an antacid, calculated to produce the desired therapeutic effect in association with the required pharmaceutical carrier. The requirements for the dosage unit forms of the invention are dictated by and directly dependent on (a) the unique characteristics of the immunoglobulin composition chosen, the antacid and the particular therapeutic effect to be achieved, and (b) the limitations inherent in the art of compounding such an immunoglobulin fraction for the treatment of autoimmune disease herein disclosed in detail.

The immunoglobulin composition with or without an antacid is compounded for convenient and effective administration in effective amounts with a suitable pharmaceutically acceptable carrier in dosage unit form as hereinbefore described. A unit dosage form can, for example, contain the Cohn Fraction II, Cohn Fraction III or Cohn Fraction II+III in amounts ranging from about 5 mg/kg to about 5 g/kg and, if desired, an antacid such as cimetidine in an amount ranging from about 200 to about 1000 mg.

Clinically observable results from the administration of Cohn Fraction II, Cohn Fraction III or Cohn Fraction II+III in conjunction with antacid may be observed immediately or as early as in 2 weeks. However, it may take up to 6 weeks or more to obtain a measurable benefit. Initial dose levels used during the first few weeks of treatment may be reduced once clinical improvement has been observed. Reductions in dose levels of up to 90% may be made after the first few weeks.

In another embodiment, the immunoglobulin composition is terminally sterilized. Specifically, a human plasma fraction such as Cohn Fraction II+III is exposed to controlled gamma irradiation at a rate sufficient to sterilize Cohn Fraction II+III for therapeutic use. Exposure is at a rate of about 2 to about 3 KGy per hour for a total dose of about 20 to about 50 KGy. Gamma irradiation is applied to capsules containing Cohn Fraction II+III precipitate to destroy or otherwise inactivate inherent viral and bacterial contaminants. Irradiating Cohn Fraction II+III capsules for a total dose of about 25 to about 50 KGy ensures a Sterility Assurance Level (SAL) of about 10⁻⁶ and does not destabilize the immunoglobulin composition contained therein. Surprisingly, the biological activity of the immunoglobulin fraction is maintained, despite the high dose of irradiation (Table 1).

TABLE 1
ANTIBODY REACTIVITY AFTER GAMMA IRRADIATION*

| DILUTION | SANDOGLOBULIN | FRACTION 2 + 3 | | |
|---|---|---|---|---|
| 1/100 | Non-irrad | Non-irrad | 12kGy | 25kGy |
| Pneuma | 1.32 | 1.28 | 1.19 | 1.18 |
| Tet-Dipth | 1.8 | 1.72 | 1.75 | 1.64 |
| Candida | 1.85 | 1.52 | 1.75 | 1.44 |
| *Data are expressed as optical density (A405 nm) by ELISA. The results represent the difference between the | | | | |
| # response to the antigens indicated (vaccines and skin test reagents) minus the response to human serum albumin. | | | | |
| # All responses are the mean of duplicate determinations. The responses to albumin were between 0.13 and 0.20. | | | | |

In another embodiment, the human plasma immunoglobulin composition is terminally sterilized by heat to destroy or otherwise inactivate inherent viral and bacterial contaminants. Heat sterilization, for example moist heat sterilization, is conventionally employed by directly contacting the composition of the immunoglobulin composition with saturated steam at temperatures ranging from about 150° C. to about 350° C. and at pressures up to 5 bar. However, the skilled artisan readily appreciates that modifications to heat sterilization are conventionally implemented in accordance with the present invention.

The oral treatment method in accordance with the present invention may be used to treat rheumatoid arthritis (including juvenile polyarticular rheumatoid arthritis) and other related immune-mediated diseases such as Still's disease, Sjogrens Syndrome, vasculitis, including Systemic Lupus Erythmatosus (SLE), peripheral neuropathy, Raynauds Phenomenon, sensory-neural hearing loss (Meniere's Disease), fibromylagia, spondyloarthopathies including, inflammatory bowel disease (ulcerative colitis, Crohn's disease and mucinous colitis), psoriatic arthritis, Reiter's Syndrome and ankylosing spondylitis, temporal arteritis, polymyalgia rheumatica and agammaglobulinemia. The treatment of spondyloarthopathies according to the present invention is contemplated to employ the same dosages as for rheumatoid arthritis and the same treatment protocol.

The invention will now be further described by the following non-limiting examples.

### EXAMPLE 1

Preparation of Human Cohn Plasma Fraction II+III

Plasma from U.S. donors was screened and tested for transmissible disease markers according to FDA and cGMP requirements. Each blood sample was screened for Anti-HIV-1/HIV-2 and HIV-1 p24, Antigen HIV-11985, HIV-2 1992, HIV Antigen 1996; Hepatitis B Surface Antigen and Hepatitis B core Antibody 1971/1987; Hepatitis C, Anti-HCV 199D; Alanine Aminotransferase Liver Enzyme, 1986; Serologic Test for Syphillus and HTLV-I/II 1988.

Fresh and/or recovered frozen plasma that had been stored at −18° C., or below, was thawed and brought to 0-2° C. After pooling, cold ethanol was added to a final concentration of 8% (vol./vol.). The mixture was kept at −1° C. to −3° C.; the pH was adjusted to 7.2-7.3. The resulting precipitate (Fraction I) was removed by centrifugation using a Sharples AS 16 with a flow rate between 600 and 750 ml per minute.

The supernatant was cooled to −5° C. and the pH was adjusted to 6.7 to 6.9 with citric acid. More cold ethanol was added to reach a final concentration of 25% (vol./vol.). This ethanol concentration maximized the recovery of IgG, IgA and IgM and also included amounts of albumin, alpha and beta globulins in the resulting Fraction II+III precipitate which was collected by centrifugation.

The Fraction II+III precipitate was suspended in 0° C.-5° C. water-for-injection (WFI), containing 1% glycine and 2% dextrose (final concentrations). The pH was adjusted to 6.0 with 0.1 M citric acid. The protein concentration of the Fraction II+III solution was 2.5 0.5%.

Fraction II+III solution (0-10° C.) was placed in containers at a solution layer depth of 0.70 0.2 inch. The solution was then freeze-dried and stored in sealed plastic containers at 1 to 10° C.

Lyophilized Fraction II+III powder in sealed plastic containers was exposed to controlled gamma irradiation. Exposure was 2-3 KGy per hour for a total of 25 to 50 KGy.

### EXAMPLE 2

### Preparation of Human Cohn Plasma Fraction II and Cohen Plasma Fraction III

Fraction II+III precipitate, prepared as set forth in Example 1, is dissolved in sufficient water-for-injection at −5° C. to give a 1% protein concentration. The pH is adjusted to 7.2 and cold ethanol is added to reach a final concentration of 20% to 25% (vol./vol.). The mixture is allowed to stand at −5° C. for 2 to 24 hours. The precipitate, Fraction II, is removed from the filtrate by centrifugation. (See Oncley et al. (1949), J. Am. Chem. Soc., 71:541-550, incorporated herein by reference). The supernatant produced contains Fraction III. The pH of the supernatant is adjusted to 5.7 and cold ethyl alcohol is added to reach a final concentration of 25% (vol./vol.) The mixture is allowed to stand at −5° C. for 2-24 hours. The precipitate, Fraction III is removed from the filtrate by centrifugation (see Oncley et al., supra).

Fraction II and/or Fraction III is redissolved in water suitable for injection to give a solution that is 1% to 5% protein and about 15% glycine. The product is then lyophilized by freezing for about 4 hours at a temperature of −30° C. to −35° C. The shelf temperature is increased from −30° C. to −10° C. for 2 hours. The shelf temperature is then increased to 0° C. The primary drying is done at 0° C. or until the thermocouples are at 0° C. (about 20 hours from start of cycle). The secondary cycle is conducted at 30° C. shelf temperature for about 6 hours.

What is claimed is:

1. A method of treating an immune-mediated disease in a patient comprising orally administering to said patient an immunoglobulin composition comprising Cohn Fraction II+III in an amount sufficient to provide a clinically observable improvement in the disease symptoms of said patient.

2. The method of claim 1 wherein the amount of immunoglobulin composition which is administered to said patient is between 5 mg/kg to 5 g/kg per day.

3. The method of claim 2 wherein the amount of immunoglobulin composition which is administered to said patient is about 1000 mg per day.

4. The method of claim 1 wherein said immunoglobulin composition is administered in a unit dosage form.

5. The method of claim 1 wherein said immunoglobulin composition is in a powdered form.

6. The method of claim 1 wherein said immunoglobulin composition is dispersed in pharmaceutically acceptable carrier.

7. The method of claim 1 wherein said immune-mediated disease is selected from the group consisting of rheumatoid arthritis, juvenile polyarticular rheumatoid arthritis, Still's disease, Sjogrens Syndrome, vasculitis, Systemic Lupus Erythmatosus, peripheral neuropathy, Raynauds Phenomenon, sensory-neural hearing loss (Meniere's Disease), fibromylagia, inflammatory bowel disease (ulcerative colitis, Crohn's disease, and mucinous colitis), psoriatic arthritis, Reiter's Syndrome, ankylosing spondylitis, temporal arteritis, polymyalgia rheumatica and agammaglobulinemia.

8. A pharmaceutical composition comprising Cohn Fraction II+III and a pharmaceutically acceptable carrier.

9. The pharmaceutical composition of claim 8 wherein said Cohn Fraction II+III is irradiated.

10. A method of treating an immune-mediated disease in a patient comprising orally administering to said patient an immunoglobulin composition comprising Cohn Fraction II in an amount sufficient to provide a clinically observable improvement in the disease symptoms of said patient.

11. The method of claim 10 wherein the amount of immunoglobulin composition which is administered to said patient is between 5 mg/kg to 5 g/kg per day.

12. The method of claim 11 wherein the amount of immunoglobulin composition which is administered to said patient is about 1000 mg per day.

13. The method of claim 10 wherein said immunoglobulin composition is administered in a unit dosage form.

14. The method of claim 10 wherein said immunoglobulin composition is in a powdered form.

15. The method of claim 10 wherein said immunoglobulin composition is dispersed in pharmaceutically acceptable carrier.

16. The method of claim 10 wherein said immune-mediated disease is selected from the group consisting of rheumatoid arthritis, juvenile polyarticular rheumatoid arthritis, Still's disease, Sjogrens Syndrome, vasculitis, Systemic Lupus Erythmatosus, peripheral neuropathy, Raynauds Phenomenon, sensory-neural hearing loss (Meniere's Disease), fibromylagia, inflammatory bowel disease (ulcerative colitis, Crohn's disease, and mucinous colitis), psoriatic arthritis, Reiter's Syndrome, ankylosing spondylitis, temporal arteritis, polymyalgia rheumatica and agammaglobulinemia.

17. A pharmaceutical composition comprising Cohn Fraction II and a pharmaceutically acceptable carrier.

18. The pharmaceutical composition of claim 17 wherein said Cohn Fraction II is irradiated.

19. A method of treating an immune-mediated disease in a patient comprising orally administering to said patient an immunoglobulin composition comprising Cohn Fraction III in an amount sufficient to provide a clinically observable improvement in the disease symptoms of said patient.

20. The method of claim 19 wherein the amount of immunoglobulin composition which is administered to said patient is between 5 mg/kg to 5 g/kg per day.

21. The method of claim 20 wherein the amount of immunoglobulin composition which is administered to said patient is about 1000 mg per day.

22. The method of claim 19 wherein said immunoglobulin composition is administered in a unit dosage form.

23. The method of claim 19 wherein said immunoglobulin composition is in a powdered form.

24. The method of claim 19 wherein said immunoglobulin composition is dispersed in pharmaceutically acceptable carrier.

25. The method of claim 19 wherein said immune-mediated disease is selected from the group consisting of rheumatoid arthritis, juvenile polyarticular rheumatoid arthritis, Still's disease, Sjogrens Syndrome, vasculitis, Systemic Lupus Erythmatosus, peripheral neuropathy, Raynauds Phenomenon, sensory-neural hearing loss (Meniere's Disease), fibromylagia, inflammatory bowel disease (ulcerative colitis, Crohn's disease, and mucinous colitis), psoriatic arthritis, Reiter's Syndrome, ankylosing spondylitis, temporal arteritis, polymyalgia rheumatica and agammaglobulinemia

26. A pharmaceutical composition comprising Cohn Fraction III and a pharmaceutically acceptable carrier.

27. The pharmaceutical composition of claim 26 wherein said Cohn Fraction III is irradiated.

28. A composition comprising Cohn Fraction II+III.
